(19) United States
(12) Patent Application Publication
(10) Pub. No.: US 2006/0069359 A1
(43) Pub. Date: Mar. 30, 2006

(54) Body fluid collection system

(76) Inventors: Joseph DiPalma, Neenah, WI (US); Cornelis J. Bosselaar, Appleton, WI (US); Daniel L. Ellingson, Appleton, WI (US)

Correspondence Address: BRINKS HOFER GILSON & LIONE, P.O. BOX 10395, CHICAGO, IL, 60610, US

(21) Appl. No.: 10/955,319

(22) Filed: Sep. 30, 2004

(51) Int. Cl. A61M 1/00

(52) U.S. Cl. 604/327

(57) ABSTRACT

A fluid collection device for collecting body fluids from a user includes a fluid director having a trough formed at least in part from a flexible material. The trough has a generally open inlet defined at least in part by a peripheral rim adapted to contact the body of the user and an outlet. A fluid receptacle is connected to the fluid director adjacent the outlet. In one embodiment, the fluid receptacle includes an absorbent material. In one aspect, the fluid director includes at least one attachment device spaced from the fluid receptacle and which is adapted to releasably secure the fluid director to at least one of the body of the user and an undergarment worn by the user.

### BACKGROUND

The present invention relates generally to body fluid collection systems, and in particular, to a body fluid director for use with a fluid receptacle.

Various absorbent products are designed to collect and absorb bodily fluids. For example, feminine pads absorb menstrual fluid, incontinence pads/guards absorb urine and diapers collect and absorb urine and feces. Often, it is desirable to locate an absorbent material close to the body to intercept and absorb the fluids more directly. However, such products can be bulky and/or uncomfortable. Other products include receptacles that channel the fluid away from the body of the user to a receptacle. Typically, however, these type of products are formed from relatively rigid materials that can be uncomfortable to the user and difficult to apply. In addition, many of these types of products require belt-like structures, or rely on a separate receptacle support system. Other products, which are held in place by the undergarment of the user, may tend to sag and/or pull away from the body of the user as they absorb fluids.

Accordingly, the need remains for a product that fits close to the body, yet is non-bulky and comfortable and which can be secured to the user separate and apart from the fluid receptacle.

### SUMMARY

Briefly stated, in one aspect, a fluid collection device for collecting body fluids from a user includes a fluid director having a trough formed at least in part from a flexible material. The material has a bending stiffness less than or equal to about 3.00 gf cm²/cm. In other embodiments, the bending stiffness is less than or equal to about 1.00 gf cm²/cm, less than or equal to about 0.50 gf cm²/cm, less than or equal to about 0.10 gf cm²/cm and less than or equal to about 0.05 gf cm²/cm. In one embodiment, the material is substantially fluid impervious. The trough has a generally open inlet defined at least in part by a peripheral rim adapted to contact the body of the user and an outlet. A fluid receptacle is connected to the fluid director adjacent the outlet. In one embodiment, the fluid receptacle includes an absorbent material.

In another aspect, the fluid director includes at least one attachment device spaced from the fluid receptacle and which is adapted to releasably secure the fluid director to at least one of the body of the user and/or an undergarment worn by the user. In one embodiment, the attachment device includes a strap, which includes an end portion having a releasable engagement portion. In other embodiments, the attachment device is formed on the trough.

In another aspect, the fluid collection device further includes an undergarment disposed on a garment side of the absorbent element. In one embodiment, the releasable engagement portion is formed on a garment side of the strap and is releasably secured to the undergarment. In another embodiment, the engagement portion is formed on a bodyside of the strap, and engages for example and without limitation the body of the user.

The various aspects and embodiments provide significant advantages over other fluid collection devices. For example and without limitation, the fluid director can be constructed separately from and without a bulky absorbent material disposed adjacent the body of the user so as to increase the comfort thereof. In addition, the director can be made from a flexible, soft material that is comfortable to the user. At the same time, the material and construction of the fluid director, in combination with the attachment mechanism, allows the fluid director to remain open and not collapse on itself.

The attachment mechanism, which may include for example and without limitation straps, provides an independent securing mechanism that can be secured to a remote portion of the body of the user, in one embodiment, without the need to accommodate release from pubic hair. The attachment device maintains a snug fit with the user's body while increasing the overall comfort of the device. In addition, when attached to the body of the user, the device can be used with a variety of undergarments, including thong underwear and the like, or by itself without the need for additional undergarments.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

FIG. 1 is a perspective view of a first embodiment of a fluid director and receptacle.

FIG. 2 is a front view of the fluid director and receptacle shown in FIG. 1 as applied to the body of a user wearing an undergarment.

FIG. 3 is a perspective view of a second embodiment of a fluid director and receptacle.

FIG. 4 is a top perspective view of a third embodiment of a fluid director and receptacle.

FIG. 5 is a cross-sectional view of the fluid director and receptacle shown in FIG. 4 taken along line 5-5.

FIG. 6 is a garment-side view of the fluid director and receptacle shown in FIG. 4.

### DETAILED DESCRIPTION OF THE PRESENTLY PREFERRED EMBODIMENTS

The term “bodyside” should not be interpreted to mean in contact with the body of the user, but rather simply means the side that would face toward the body of the user, regardless of whether an undergarment is actually being worn by the user and regardless of whether there are or may be intervening layers between the component and the body of the user. Likewise, the term “garment side” should not be interpreted to mean in contact with the garments of the user, but rather simply means the side that faces away from the body of the user, and therefore toward any outer garments that may be worn by the user, regardless of whether the undergarment is actually being worn by a user, regardless of whether any such outer garments are actually worn and regardless of whether there may be intervening layers between the component and any outer garment.

The phrases “removeably attached,” “removeably attaching,” “removeably connected,” “removeably engaged,” “releasably attached,” “releasably connected,” or “releasably engaged,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the absorbent garment.

The phrases “fixedly secured,” “fixedly engaged,” “fixedly attached,” “fixedly connected,” and variations thereof, refers to two or more elements being connected or connectable such that they are not disconnected or otherwise separated, and are not intended to be separated or disconnected, during the normal operation and use of the absorbent garment.

The terms “connecting,” “coupled,” “attached,” and “secured,” and variations thereof, broadly covers two or more items being directly connected one to the other, or indirectly by way of one or more intervening members or components.

The term “longitudinal” as used herein means the direction 500 or path running generally from the front to the back of the user. The term “lateral” as used herein means the direction 502 running generally from one side of the user to the other side of the user, and is generally substantially perpendicular to the longitudinal direction.

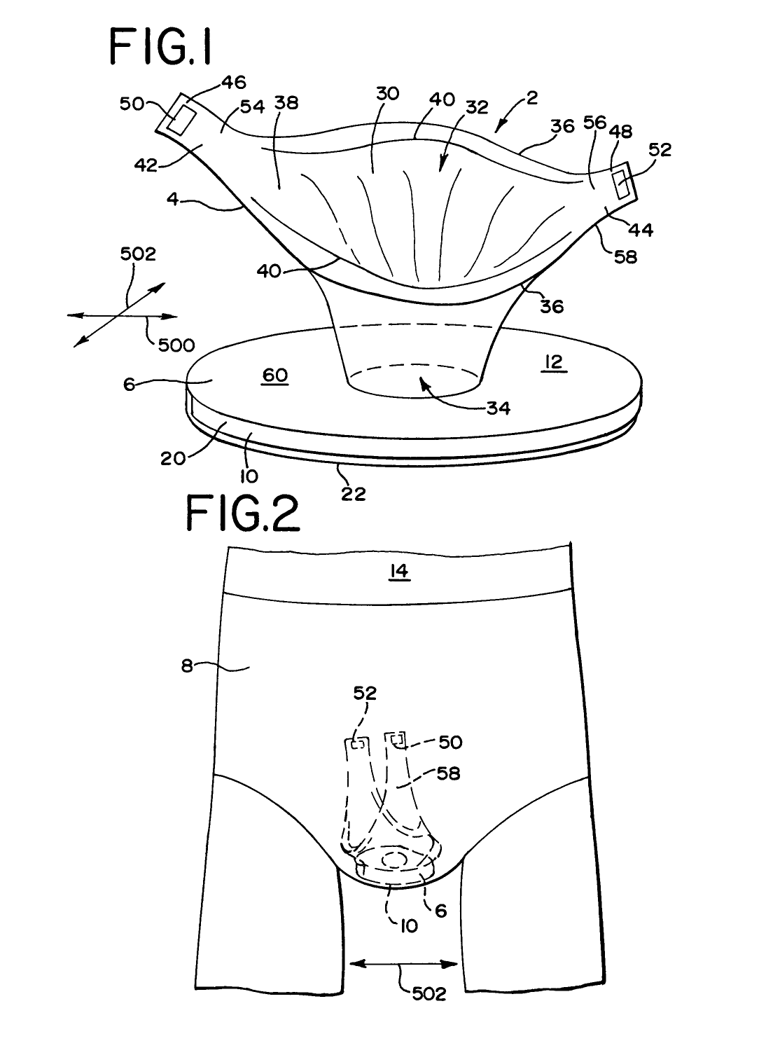

Referring to FIG. 1, a fluid collection device 2 is shown as including a fluid director 4 connected to a fluid receptacle 6. The fluid receptacle 6 is shown as an absorbent article, and in particular an absorbent pad. In other embodiments, the fluid receptacle can be configured as a non-absorbent bag, such as a catheter bag, or other collection container. As shown in FIG. 2, the absorbent article is positioned in and retained 10 by an undergarment 8, shown as a panty, in the crotch region thereof. Referring to FIGS. 1 and 2, the panty supports the garment side 10 of the pad, while a bodyside 12 thereof faces a user 14. The absorbent article includes an absorbent material configured as a liquid retention portion 20 and a liquid-impervious backsheet 22 to prevent leakage. In some embodiments, a top sheet or liner is provided on the body side of the retention portion to provide greater comfort to the user.

In one embodiment, shown in FIGS. 1 and 2, the fluid director includes a trough 30 having a generally open inlet 32 and an outlet 34. The inlet 32 is defined at least in part by a peripheral rim 36 that is adapted to contact the body of the user. It should be understood the terms “peripheral” and “periphery” refer to elements that may be continuous or discontinuous. The trough is formed by a flexible wall 38 that extends around and defines a periphery of the fluid director. In other embodiments (FIG. 4), the wall 138 does not define a circumferential enclosure, but rather is open along one or more sides. Referring to the embodiment of FIGS. 1 and 2, the trough 30 narrows such that the inlet 32 is generally larger than the outlet 34. In one embodiment, the trough has a frusto-conical shape. In other embodiments, the inlet is substantially the same size or is smaller than the outlet. In one embodiment, the inlet 32 portion of the trough, or upper receiving portion, is sufficiently large enough to enclose the part of the body generating the fluid insult, including for example and without limitation urine, menstrual fluid and feces. For example, the inlet portion defines an opening having a lateral width of between about and including 3 to 10 cm, and a longitudinal length of between about and including 5 to 30 cm.

In one embodiment, the peripheral rim 36 includes elastic members 40 extending longitudinally therealong such that the rim conforms to the body of the user. In one embodiment, the elastic members are made of Lycra® strands.

As shown in FIGS. 1 and 2, a pair of straps 42, 44 extend from the trough 30 on opposite ends thereof adjacent the inlet. The straps are integrally formed with the trough material, although it should be understood that they can be configured as separate elements that are secured to the trough. As shown in FIGS. 1 and 2, the straps 42, 44 define a portion of the periphery of the trough at the inlet end thereof and are spaced from the outlet 34. A free end portion 46, 48 of each strap includes an attachment device 50, 52 or element. In various embodiments, the straps 42, 44 can be configured with a plurality of attachment devices on the same side of the strap, or on different sides.

In one embodiment (FIG. 1), the attachment device 50, 52 is formed on a bodyside surface 54, 56 of the strap and is configured to engage and be attached to the body 14 of the user, preferably at the front or back of the user. In one embodiment, where the attachment device engages the body of the user on the front side, the strap 42 is of sufficient length such that the attachment device can be engaged with the body above the line of pubic hair, such that complexities associated with releasing from such hair are avoided.

In another embodiment (FIG. 2), the attachment device 50, 52 is formed on a garment side surface 58 of the strap and is configured to engage and be attached to the garment 8 worn by the user, for example an undergarment holding the absorbent article. In yet another embodiment, one or more of the straps are configured with attachment devices on both the bodyside and garment side surfaces thereof, such that the strap(s) can be secured to either the body of the user or a garment, or to both simultaneously. Of course, it should be understood that each strap can be configured with a different attachment device, and with attachment devices located on different surfaces (bodyside/garment side) of the straps. In various embodiments, the attachment device 50, 52 includes a releasable engagement portion, such as a releasable adhesive (including body adhesives that engage the body of the user or adhesives that engage an undergarment), hook and loop fastener, hooks, snap, button, etc. that releasably engages one or both of the body of the user or undergarment. One suitable body adhesive is disclosed in U.S. Pat. No. 6,620,143, assigned to Kimberly-Clark Worldwide Inc., the same assignee as the present application, and which is hereby incorporated herein by reference.

In other embodiments, the attachment device 50, 52 can be formed or disposed on any part of the trough, including for example and without limitation the rim portion 36 thereof. In such embodiments, the fluid director may be configured without straps, although it should be understood that the attachment device can be formed or disposed on various portions of the trough and additionally on straps or other extensions. In any of the embodiments, the attachment device can be configured on the body side or garment side surface of the trough, so as to engage respectively the body of the user or an undergarment worn by the user.

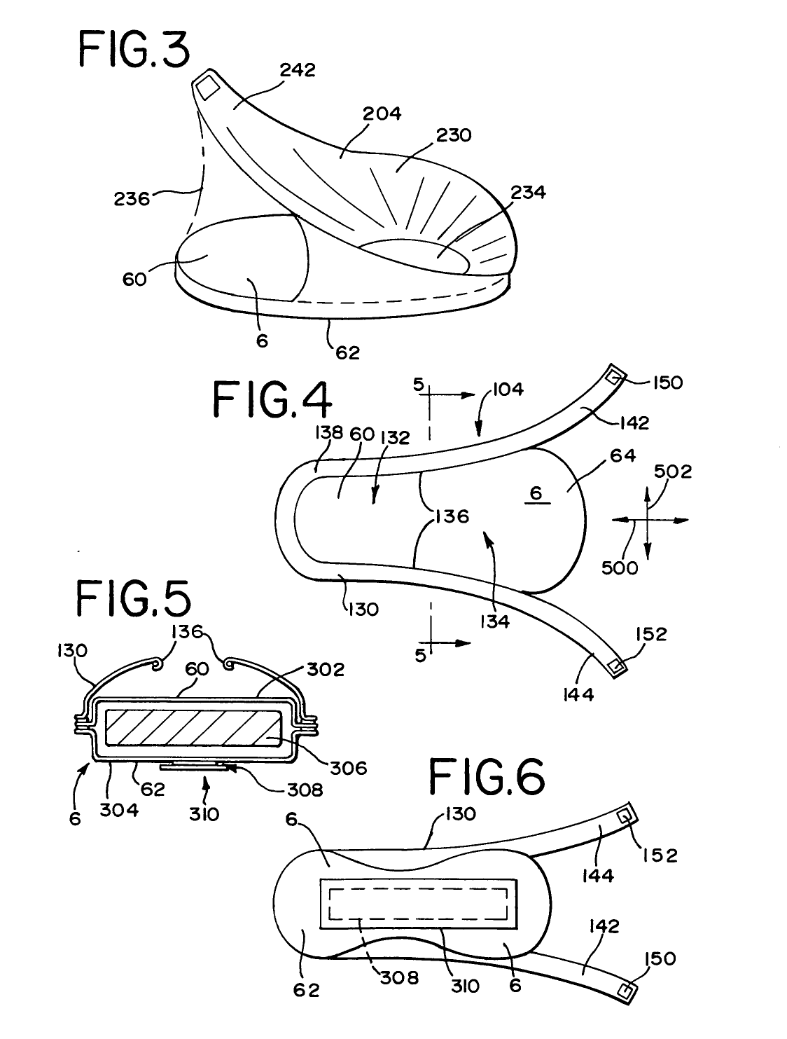

Referring to FIG. 3, an alternative embodiment of a fluid director 204 includes only one strap portion 242, which can be engaged with the body 14 of the user or the undergarment 8 (or both), on one of the front and rear sides of the user. As shown in FIG. 3, a trough 230 is secured to only a portion of the absorbent element, with the outlet 234 having a cross-sectional area less than the surface area of the top (bodyside surface) surface 60 of the absorbent element 6. Alternatively, as illustrated by phantom line 236, the trough outlet 234 may be substantially the same size as or conform to the entirety of the bodyside surface 60 of the absorbent element. In one embodiment, the absorbent element 6 is enclosed by the trough 204 (or shroud), which wraps around the absorbent element 6 on the garment side thereof. In this embodiment, the cross-sectional area of the trough or shroud immediately above the body side surface 60 of the absorbent element 6 is construed as the outlet portion 234 of the trough.

Referring to the embodiment of FIGS. 4-6, the trough 130 is not enclosed on all sides (i.e., the trough does not form a circumferential boundary). Rather, the trough 130 surrounds an absorbent element 6 on three sides and has a pair of laterally spaced straps 142, 144 extending in the generally the same longitudinal direction 500 from one end of the trough. The straps 142, 144 each include an attachment device 150, 152 that can be secured to the body of the user or an undergarment as explained above. In one desired embodiment, the straps 142, 144 extend toward the front of the user such that they are more easily accessed by the user and can be applied to the front of the body of the user, for example to the torso above the pubic hair of the user. In one embodiment, the trough surrounds the absorbent element such that a portion thereof is disposed on a garment side 62 of the absorbent element. As shown in the embodiment of FIGS. 5 and 6, the trough 130 is secured to the absorbent element along the sides thereof, for example and without limitation with bonds (e.g., adhesive, sonic and/or thermal), sewing or various mechanical fasteners. For example, in one embodiment, the trough 130 is bonded to the sides of the backsheet 304 and topsheet 302 along the sides of a retention portion 306.

In another embodiment, the trough includes two separate (not directly connected) portions, for example and without limitation two straps, that are secured to the sides of receptacle. In this embodiment, the outlet is defined as the space between the two portions immediately above the receptacle, for example and without limitation an absorbent article, while the inlet is defined by the space between the edges of the two portions proximate the body of the user.

In this embodiment, the inlet 132 is defined by the peripheral rim 136 of the trough, although the inlet is not closed on one side (i.e. is open adjacent one end 64 of the absorbent element 6). The outlet 134 is defined as the cross-section of the trough 130 immediately above the absorbent element as defined by the trough, with the outlet also being open on one end adjacent the end 64 of the absorbent element 6. The trough 130 acts as a cuff, which gaskets against the body of the user, while the rear portion of the trough forms a pocket and functions as a dam. The peripheral rim 136 can be provided with elastic elements to improve the fit of the trough against the body of the user.

In other embodiments (not shown), the outlet of the director is adapted to be connected to a non-absorbent fluid receptacle, such as a catheter bag. In these embodiments, the outlet can be connected to the fluid receptacle with a tube or other connector. In this embodiment, the fluid receptacle does not need to be maintained in the crotch region of the user, but rather can be remotely located, or attached to another part of the body, such as the leg of the user.

The trough 30, 130, 230 is formed of a thin, flexible material, for example and without limitation lightweight non-woven materials, paper fabrics such as spunbond or tissue, and/or combinations thereof. The thickness of the material in various embodiments is less than or equal to about 1.00 mm, less than or equal to about 0.50 mm, less than or equal to about 0.10 mm or less than or equal to about 0.050 mm. Preferably, the material is substantially fluid impermeable such that it captures and directs any bodily fluids to the absorbent element 6 or other fluid receptacle. In various embodiments, the material is considered substantially fluid impermeable even though vapors can pass therethrough or fluid can be forced therethrough under pressure. The material may be treated with various fluid repellant materials.

The material is preferably breathable so as to maintain comfort and skin wellness for the user. In various embodiments, the entirety of the trough material can be elasticized, such as by being formed of a stretch bonded laminate (SBL) material. Alternatively, as explained above, the material can be elasticized in specific discrete locations by incorporating strands of elastic material. The thin flexible material of the trough maximizes the comfort to the user as well as minimizes the packaging volume. In addition, the construction avoids the need for rigid or semi-rigid components or additional supporting components. At the same time, the trough configuration ensures that the fluid passageway defined between the inlet and outlet remains open, without the fluid director collapsing on itself to close off the fluid flow to the fluid receptacle.

The term “flexible” as used herein means the material has a bending stiffness less than or equal to about 3.00 gf cm²/cm as measured using the Kawabata Pure Bending Stiffness test, which is explained herein. The testing set forth herein is performed where the material samples are conditioned 24 hours and tested under TAPPI standard conditions of 23±1° C. and 50±2% RH. The test equipment discussed is exemplary and should be used to conduct the testing. However, alternative equipment that is equivalent in all material respects for the given test can be used also (but in the event of conflict between test results the test results from the exemplary equipment shall control). In particular, the Kawabata bending stiffness test uses a KES-FB2 pure bending tester, which is commercially available from Kato Tech Co., Ltd. 26 Karato-Cho, Nishikujo Minami-Ku, Kyoto 601 Japan.

Samples of the trough material to be tested are prepared by cutting a portion of the material to size, which size may vary depending on availability and could range from 1 cm to 20 cm in length and at least 1 cm in width to allow for proper clamping of the sample between the front and back chucks of the equipment. The sample is not limited to a square shape and could be a rectangular shape. Ultimately, the data is normalized by the length of the sample on per a centimeter basis, so size is not a determinative factor in the test. The Bending Tester bends the sample in the range of curvatures of ±2.5 cm⁻¹ at a constant rate of 0.5 cm⁻¹/sec. The bending stiffness is defined as the mean of slopes of plots of bending moment (with a unit of gf cm²/cm) vs. curvature (cm⁻¹) when the sample is bent in both sides (i.e. wire and anvil sides). For the purpose of calculation of slopes, the curvature between 0.5 and 1.5 cm⁻¹ is considered as the forward bending of the face side (e.g. wire side) of the sample while the curvature between −0.5 and −1.5 cm⁻¹ is considered as the backward bending of the other face side (e.g. anvil side) of the sample. However, it is not necessary to have the forward or backward bending associated with a given side of the sample (i.e. wire or anvil side).

In the table below, the samples were tested in the MD direction. However, since the MD and CD of a product is not always easily identified, each sample may be tested as above in one direction and then the other direction which is perpendicular to the first direction relative to a two dimensional plane of the surface of the sample. Three samples are tested and the bending stiffness of both directions (e.g., MD and CD, or their equivalents if not known per the perpendicular orientation set forth here) in grams force cm²/cm (gf cm²/cm) is added together and divided by two and reported as the average bending stiffness for each sample. The average bending stiffnesses of the three samples are added together and the collective total average bending stiffness is divided by 3, which thereby determines the Bending Stiffness of the trough material, which is discussed herein and set forth in the claims.

As set forth in Table 1, the bend stiffness for various materials was determined.

TABLE 1
Kawabata Pure Bending Stiffness

| | | | | | Bending | |
|---|---|---|---|---|---|---|
| | | 0.4 mm ctr | | | Stiffness | |
| Sample | Material | (and adj. | Sample | | (gf * cm2/cm) | |
| ID | (Actual basis wt.) | plate used) | width (cm) | Sensitivity | Mean (n = 3) | std. dev. |
| C | 0.7 mil PE Film (18 gsm) | Y | 10 | 2 × 5 | 0.0044 | 0.0023 |
| A | bonded/carded web | Y | 10 | 2 × 5 | 0.0221 | 0.0007 |
| | (17 gsm) | | | | | |
| B | 0.6 osy spunbond (21 gsm) | Y | 10 | 2 × 5 | 0.0393 | 0.0068 |
| F | 2.7 mil LDPE film (62 gsm) | Y | 10 | 2 × 5 | 0.1098 | 0.0107 |
| D | nonwoven wing material | Y | 10 | 2 × 1 | 0.1430 | 0.0127 |
| | (56 gsm) | | | | | |
| H | Kotex peel strip paper (44 gsm) | Y | 10 | 2 × 1 | 0.5016 | 0.0583 |
| E | 1.8 osy spunbond (63 gsm) | Y | 10 | 2 × 1 | 0.6133 | 0.0663 |
| I | KC Rec Fab (159 gsm) | No | 1 | 5 × 1 | 2.1797 | 0.1946 |
| G | writing paper (79 gsm) | Y | 5 | 5 × 1 | 3.0004 | 0.2036 |
| J | Blotter paper (313 gsm) | No | 1 | 5 × 1 | 10.0772 | 1.3020 |

Constant for all samples:
Samples length=10 cm
Number of plies=1
Repetition=1
Maximum Curvature Value=Set
Curvature Value Range=0.5-1.5
Curvature/Hysteresis=1.0
Measurement Mode=One cycle

In reviewing the data, samples A-I had satisfactory bending stiffness values, with the samples A-F and H having more desirable bending stiffness values and with samples A-C having the lowest bending stiffness values. As such, the material desirably has a bending stiffness less than or equal to about 3.00 gf cm²/cm, more desirably less than or equal to about 1.00 gf cm²/cm, more desirably less than or equal to about 0.50 gf cm²/cm, more desirably less than or equal to about 0.10 gf cm²/cm and more desirably less than or equal to about 0.05 gf cm²/cm.

The trough material can be formed as a composite, or laminate material, otherwise referred to as substrates or laminates, with an elastic core sandwiched therebetween. In one embodiment, the elastic core is made of an elastomeric film or nonwoven elastic or stretchable material including for example block copolymers of polystyrene, polyisoprene or polybutadiene, copolymers of ethylene, natural rubbers, urethanes, kratons, and co-extrusions/blends of the aforementioned material. The elastic core can be formed as a membrane or from a plurality of elastic strands, as described above. In one embodiment, two or more layers are bonded to the elastic core, and/or each other, with various adhesives, such as hot melt, or by other techniques, including for example and without limitation ultrasonic bonding and heat pressure sealing. In one embodiment, the two layers are made of a non-woven material such as a spunbond material, a bonded carded material or other known materials. In this way, the trough material panels are made of a stretchable/elastic material.

As used herein, the interchangeable terms “stretchable” and “elastic,” and variations thereof, refer to a material that can elongate or deform (stretch) in response to the application of a tensile force, and upon removal of the tensile force the material can retract and become shorter. Because of hysteresis, the material may not be able to fully recover or return to its original, pre-stretched length. Thus, a stretchable or elastic material can be stretched and upon relaxing the material, will tend to resume its original shape.

It should be understood that the trough can be made of a single layer or substrate of non-woven material, a bi-layer substrate made of non-woven materials without an elastic core, or more than two layers or substrates. Of course, it should be understood that other knitted or woven fabrics, non-woven fabrics, elastomeric materials, polymer films, laminates and the like can be used to form the trough. The term “non-woven” web or material, as used herein, means a web having a structure of individual fibers or filaments that are interlaid, but not in an identifiable manner and without the aid of textile weaving or knitting, as in a knitted or woven fabric.

Examples of elastomeric composite materials for use in the trough include a continuous filament stretch bonded laminate (CFSBL), a vertical filament laminate (VFL), neck-bonded-laminate (NBL), a stretch-bonded-laminate (SBL), a necked-stretch bonded laminate (NSBL) or a necked-thermal laminate, or the like, as well as combinations thereof. Exemplary CFSBL, NBL, SBL, and NSBL materials are described in U.S. Pat. Nos. 5,226,992, 4,981,747, 4,965,122, 5,336,545, 5,385,775, 5,414,470, 4,720,415, 4,789,699, 4,781,966, 4,657,802, 4,652,487, 4,655,760, 5,116,662 and 5,114,781, and 6,323,389, all of which are hereby incorporated herein by reference. Exemplary VFL materials are described in U.S. Provisional Patent Application Ser. No. 60/204,307, filed May 15, 2000 and entitled “Method and Apparatus for Producing Laminated Articles,” and PCT application WO 01/88245 A2, both assigned to Kimberly-Clark Worldwide, Inc., the Assignee of the present application, with the entire disclosures of both being hereby incorporated herein by reference. Such laminates can provide an improved combination of cloth-like feel and elastomeric stretchability. The trough can be composed of materials that are elastic or elastomeric and exhibit biaxial stretch characteristics or lateral/longitudinal stretch characteristics, or which are extensible composites.

In one embodiment, the trough is made of breathable barrier material, including for example a highly breathable stretched thermal laminate material. One suitable material is disclosed for example and without limitation in U.S. Pat. No. 6,309,736, assigned to Kimberly-Clark Worldwide, Inc., the same assignee as the present application, the entire disclosure of which is hereby incorporated herein by reference.

It should be understood that the term “absorbent article,” or variations thereof, refers to any material or assembly capable of absorbing liquids or bodily exudates, and may be formed from a single material or component, for example a retention portion, or can be formed as a composite of several components. For example, various suitable absorbent articles include without limitation Kotex® pads/liners and Poise® pads, both available from Kimberly-Clark Corporation, Neenah, Wis.

As explained above, and with reference to FIG. 5, the absorbent article 6 can include a top sheet 302, backsheet 304 (shown as a baffle) and retention portion 306, all of which can be joined for example with adhesive bonds, sonic bonds, thermal bonds, pinning, stitching or any other attachment techniques known in the art, as well as combinations thereof. In one embodiment, one or more elastic members are sandwiched between the top sheet and backsheet along the side edges thereof. Additional layers, including for example, a liquid acquisition and distribution layer, also referred to as a surge or transfer layer, may also be incorporated into the absorbent article. In one embodiment, wherein the absorbent article is formed as a pad, as shown in FIG. 6, the garment side 64 of the absorbent article further includes an attachment device 308, including for example an adhesive, that can be secured to the user's undergarment once a protective peel strip 310 is removed.

The retention portion is made of an absorbent material, which can be any material that tends to swell or expand as it absorbs exudates, including various liquids and/or fluids excreted or exuded by the user. For example, the absorbent material can be made of airformed, airlaid and/or wetlaid composites of fibers and high absorbency materials, referred to as superabsorbents. Superabsorbents typically are made of polyacrylic acids, such as FAVOR 880 available from Stockhausen, Inc. of Greensboro, N.C. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. In addition, various foams, absorbent films, and superabsorbent fabrics can be used as an absorbent material. Various acceptable absorbent materials are disclosed in U.S. Pat. No. 5,147,343 for Absorbent Products Containing Hydrogels With Ability To Swell Against Pressure, U.S. Pat. No. 5,601,542 for Absorbent Composite, and U.S. Pat. No. 5,651,862 for Wet Formed Absorbent Composite, all of which are hereby incorporated herein by reference. Furthermore, the proportion of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom. The retention portion preferably can be made of a single or dual layer of absorbent material.

In operation, and with reference to FIGS. 1-6, the outlet portion 34, 234, 134 of the fluid director is secured to the fluid receptacle, for example and without limitation by enveloping a portion (or an entirety) of the absorbent material with the trough adjacent the outlet 134, 234 (FIGS. 3 and 4), connecting a tube to the outlet (not shown), or securing the outlet to a top surface of the fluid receptacle (FIGS. 1-2), for example by bonding, including adhesive bonds, sonic bonds, thermal bonds, pinning, stitching or any other attachment techniques known in the art, as well as combinations thereof. In other embodiments, where the fluid director surrounds and envelopes the fluid receptacle, the fluid director can be further connected to the fluid receptacle with bonding or other attachment techniques or the fluid receptacle can be connected to the fluid director simply by being pocketed therein, without any other connection mechanism.

In one embodiment (FIGS. 1-6), the fluid director 4, 104, 204 and fluid receptacle 6 are positioned in the undergarment 8, which includes for example and without limitation a panty, an absorbent garment (such as an adult incontinence garment) or even the outermost garments of the user if worn next to the body of the user. One or both strap portions 42, 44, 142, 144, 242 of the fluid director, or other attachment devices of the fluid director if no straps are included, are then independently secured to the undergarment or the body of the user, depending on the particular configuration of the straps and the desired usage as explained above. The attachment devices, in combination with the construction of the fluid director, provide a snug, comfortable fit with the body of the user.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

What is claimed is:

1. A fluid collection device for collecting body fluids from a user, the collection device comprising:
a fluid director comprising a trough formed at least in part from a flexible material, wherein said material has a bending stiffness of equal to or less than about 3.00 gf cm²/cm, said trough having a generally open inlet defined at least in part by a peripheral rim adapted to contact the body of the user and an outlet; and
a fluid receptacle connected to said fluid director adjacent said outlet.

2. The fluid collection device of claim 1 wherein said material has a thickness of less than 0.50 mm.

3. The fluid collection device of claim 1 wherein at least a portion of said material is elasticized.

4. The fluid collection device of claim 1 wherein said fluid receptacle comprises an absorbent material.

5. The fluid collection device of claim 4 wherein said fluid director envelopes at least a portion of said absorbent material at said outlet.

6. The fluid collection device of claim 4 wherein said fluid director comprises an attachment device spaced from the connection of said fluid receptacle and said fluid director adjacent said outlet, said attachment device adapted to releasably secure the fluid director to at least one of the body of the user and an undergarment worn by the user.

7. The fluid collection device of claim 6 wherein said attachment device comprises a strap having an end portion comprising an engagement mechanism.

8. The fluid collection device of claim 7 wherein said engagement mechanism comprises a releasable adhesive.

9. The fluid collection device of claim 1 wherein said peripheral rim comprises an elastic material.

10. The fluid collection device of claim 1 wherein said bending stiffness is less than or equal to about 1.00 gf cm²/cm.

11. The fluid collection device of claim 10 wherein said bending stiffness is less than or equal to about 0.05 gf cm²/cm.

12. The fluid collection device of claim 1 wherein said flexible material is substantially fluid impervious.

13. A fluid collection device for collecting body fluids from a user, the collection device comprising:
an absorbent element comprising an absorbent material and a fluid impervious backsheet; and
a fluid director comprising a trough formed at least in part from a flexible material, said trough having a generally open inlet defined at least in part by a peripheral rim portion adapted to contact the body of the user and an outlet, said fluid director connected to said absorbent element adjacent said outlet, said fluid director further comprising at least one attachment device spaced from said absorbent element and adapted to releasably secure said fluid director to at least one of a body of the user and an undergarment worn by the user, said at least one attachment device comprising a releasable engagement portion.

14. The fluid collection device of claim 13 wherein said at least one attachment device comprises a strap having an end portion configured with said releasable engagement portion.

15. The fluid collection device of claim 14 further comprising an undergarment disposed on a garment side of said absorbent element, wherein said releasable engagement portion is formed on a garment side of said strap and is releasably secured to said undergarment.

16. The fluid collection device of claim 14 wherein said releasable engagement portion is formed on a body side of said strap.

17. The fluid collection device of claim 13 wherein said flexible material has a bending stiffness less than or equal to about 1.00 gf cm²/cm.

18. The fluid collection device of claim 13 wherein at least a portion of said flexible material is elastic.

19. The fluid collection device of claim 13 wherein said director envelopes at least a portion of said absorbent element at said outlet.

20. The fluid collection device of claim 13 wherein said peripheral rim comprises an elastic material.
